(19) United States
(12) Patent Application Publication
(10) Pub. No.: US 2022/0054742 A1
(43) Pub. Date: Feb. 24, 2022

(54) PORTABLE INFUSION PUMP WITH PINCH/SQUEEZE PUMPING ACTION

(71) Applicant: Modular Medical, Inc., Escondido, CA (US)

(72) Inventors: David Neese, Escondido, CA (US); Paul M. DiPerna, Escondido, CA (US); Marc D. Goldman, San Diego, CA (US)

(21) Appl. No.: 17/001,330

(22) Filed: Aug. 24, 2020

(51) Int. Cl. A61M 5/142; A61M 5/145

(52) U.S. Cl. CPC A61M 5/14216; A61M 2205/8275; A61M 5/14244; A61M 5/1452

(57) ABSTRACT

A device for infusing a medicament into a patient includes a disposable component having a collapsible reservoir for holding the medicament, a cannula, and an elastomeric tube connected in fluid communication between the reservoir and the cannula. A manipulator is mounted on a chassis for engagement with the elastomeric tube. The manipulator includes a piston and respective pinchers upstream and downstream from the piston for a close/open open/close changeover operation. In concert therewith, the piston cyclically advances and withdraws relative to a platen to thereby alternatingly dilate/constrict the tube section. When the upstream pincher is open, the piston withdraws to create a low fluid pressure, p_{Lo}, in the tube section to draw medicament into the tube. Alternately, when the downstream pincher is open, the piston is advanced to create a high fluid pressure, p_{Hi}, on the tube to infuse medicament into the patient.

### FIELD OF THE INVENTION

The present invention pertains generally to medical devices that infuse liquid medicaments into a patient. More specifically, the present invention pertains to disposable components that define a fluid pathway which is engageable with a force generating mechanism. In turn, the force generating mechanism manipulates the disposable component to move fluid through the pathway without invading the pathway. The present invention is particularly, but not exclusively useful as a disposable component for an insulin pump that includes an elastomeric tube, where the forces for moving insulin through the tube are externally exerted only against the tube.

### BACKGROUND OF THE INVENTION

Medical pumps for the infusion of fluid medicaments into a patient must necessarily serve a particular purpose, and be specifically designed to facilitate and accomplish that purpose. Paramount considerations for the design of a medical infusion pump must focus on several patient-specific needs that must be addressed. For example, these considerations require attention to such factors as: the particular drug to be infused, a proper drug dosage, an infusion protocol, any clinically prescribed regimen, the ability to create and maintain sterile operating conditions, patient convenience, and overall operational reliability; to name but a few.

In the specific case where the patient is diabetic, and needs to follow a prolonged medication regimen which requires operational control by the patient over the process, the above noted considerations are particularly important. Moreover, for a diabetic patient, an infusion pump that can be easily, safely, and efficiently operated by the patient is essential. To ensure these capabilities, a mechanically simple infusion pump is essential.

In light of the above, it is an object of the present invention to provide a component for a portable medical fluid pump that can be easily replaced and exchanged or disposed of after each use. Another object of the present invention is to provide a medical infusion pump where the integrity of a fluid pathway through the disposable component is never invaded or compromised by the pumping mechanism itself during its operation. Still another object of the present invention is to provide a disposable component for a pump wherein medical fluid is moved along a pathway through an elastomeric tube by only a physical manipulation of the tube. Yet another object of the present invention is to provide a disposable component for a portable medical fluid pump that is simple to manufacture, is easy to use and is relatively cost effective.

### SUMMARY OF THE INVENTION

A portable pump for infusing a fluid medicament into a patient includes a chassis with a pumping mechanism (i.e. a manipulator) which is mounted on the chassis. Further, a disposable component is provided which can be engaged with the manipulator to establish the infusion pump for the present invention.

For its combination with the manipulator, the disposable component includes: a reservoir for holding the fluid medicament; an elastomeric tube which has one end connected in fluid communication with the reservoir; and a cannula which is connected in fluid communication with the other end of the elastomeric tube. In use, the elastomeric tube of the disposable component is engaged with the manipulator on the chassis. The manipulator is then operated to exert motive forces against the elastomeric tube. It is these motive forces together with elastomeric forces of the tube that move the fluid medicament from the reservoir to the cannula for infusion into the patient.

Important considerations for material properties of the elastomeric tube are that in an unbiased state, the lumen of the tube is open and dilated. On the other hand, when the tube is pinched or squeezed by external forces, the lumen becomes closed and is constricted. However, when these external forces are relieved, the tube has sufficient memory resilience to return to its unbiased state with an open lumen.

Structurally, the manipulator includes an elongated platen that is mounted on the chassis. Opposite the platen, across an elongated receiver channel from the platen, elements of the manipulator are aligned with each other. In a downstream direction, the manipulator elements are: an upstream pincher mounted on the chassis and located adjacent the reservoir; a piston; and a downstream pincher mounted on the chassis and located adjacent the cannula. Between the reservoir and the cannula, a section of the elastomeric tube is positioned in the receiver channel for interaction with the manipulator during an operation of the infusion pump.

During an operation of the infusion pump, elements of the manipulator cooperate with each other in accordance with a predetermined sequence. Specifically, the upstream and downstream pinchers interact with each other in a close/open and open/close changeover operation. In this changeover operation the pinchers individually occlude the elastomeric tube against the platen. Also, the piston cooperates with the pinchers to cyclically exert a pumping action against the section of elastomeric tube that is positioned in the receiver channel.

In detail, an operational duty cycle begins from a start configuration of the manipulator wherein the piston and both pinchers occlude the elastomeric tube. From this start configuration, a duty cycle begins when the piston is completely withdrawn from the platen. At this time, the downstream pincher remains closed and the upstream pincher is opened. With this configuration, the elastomeric tube section in the receiver channel expands to an open lumen configuration. As this occurs, a lowered pressure, p_{Lo}, is created in the tube lumen to draw fluid medicament into the tube section from the reservoir. Once the section of the elastomeric tube has been filled, both the upstream and downstream pinchers execute a changeover operation, i.e. the upstream pincher is closed and the downstream pincher is opened.

After a changeover operation, with the downstream pincher now opened and the upstream pincher closed, the piston is incrementally advanced to constrict the lumen of the tube section. This action creates a high pressure p_{Hi} in the tube section to pump fluid medicament from the tube section, and through the cannula for infusion into the patient. The duty cycle is then ended when the piston again fully occludes the elastomeric tube section, and both pinchers are closed.

In accordance with the above disclosure, a timeline for operating the manipulator during a duty cycle will extend over a time duration t₁₋₄. Configurations for the manipulator during this timeline are:
t₀-t₁ is a time interval when the piston occludes the elastomeric tube and both the upstream and the downstream pinchers are closed to begin a duty cycle;
t₁-t₂ is a time interval when the downstream pincher remains closed, while the upstream pincher remains open and the piston is withdrawn to create a lower pressure p_{Lo} in the lumen that draws fluid medicament into the tube through the upstream end of the tube;
t₂ is a time for the upstream and downstream pincher changeover operation;
t₂-t₃ is a time interval when the upstream pincher is closed and the downstream pincher is open as the piston is advanced to create a higher pressure p_{Hi} in the lumen to expel fluid medicament from the tube through the downstream end of the tube;
t₃-t₄ is a time interval when both the upstream and the downstream pinchers are closed to end a duty cycle; and
t₄=t₀ to begin a new duty cycle.

Structurally, in a preferred embodiment for the manipulator, a camshaft is mounted on the chassis for rotation with a rotational velocity w around an axis parallel to the receiver channel. Further, three cams are mounted for rotation together on the camshaft. These cams are: a piston cam to actuate the piston; an upstream cam to actuate the upstream pincher; and a downstream cam to actuate the downstream pincher. The interaction of these cams with the section of elastomeric tubing in the receiver channel will be best appreciated by considering a two-phase operation that includes a filling operation during a time interval t₁-t₂ and a pumping operation during a time interval t₂-t₃.

For a filling operation in the time interval t₁-t₂, the upstream cam opens the upstream pincher, and the downstream cam closes the downstream pincher. The piston cam then allows the piston to be withdrawn from the tube while the section of elastomeric tube in the receiver channel expands to become dilated. Thus, as the section of elastomeric tube in the receiver channel expands, it creates the lower pressure p_{Lo}, that draws fluid medicament from the reservoir.

At the time t₂, after the section of elastomeric tube has been filled, there is a changeover operation of the pinchers and a pumping operation begins for the time interval t₂-t₃. Within the time interval t₂-t₃ the upstream cam has closed the upstream pincher, and the downstream cam has opened the downstream pincher. The piston cam then incrementally advances the piston toward the tube, to thereby pump (push) fluid medicament from the elastomeric tube for infusion through the cannula and into the patient. A torque motor which is mounted on the chassis and engaged with the camshaft simultaneously rotates the three cams during a duty cycle.

Additionally, it is to be appreciated that during an operation of the present invention p_{Hi} is greater than p_{Lo} (p_{Hi}>p_{Lo}) and the difference between p_{Hi} and p_{Lo} is less than 5 psi (p_{Hi}-p_{Lo}<5 psi). Also, each duty cycle will preferably have a time duration that is in the range of 1-5 minutes, and the fluid medicament will be insulin.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a perspective view of a disposable component adapted for engagement with a manipulator for infusing a fluid medicament into a patient;

FIG. 2 is a perspective view of the manipulator;

FIG. 3 is a perspective view of the manipulator mounted on a chassis for receiving a section of elastomeric tube from the disposable component in its receiver channel;

FIG. 4A is a cross section view of the elastomeric tube of the disposable component as seen along the line 4-4 in FIG. 1 when the tube is engaged with the manipulator to occlude the tube at the beginning, and at the end of a duty cycle;

FIG. 4B is a cross section view of the elastomeric tube as seen in FIG. 4A when the tube is configured by the manipulator to receive fluid medicament from the source of fluid medicament;

FIG. 4C is a cross section view of the elastomeric tube as seen in FIG. 4A when the tube is configured by the manipulator to infuse fluid medicament from the tube and into a patient;

FIG. 5 is a perspective view of a cam drive with cross hatched lines indicating the peripheral portions that are removed from each of three otherwise circular cams to provide the necessary cam action for an operation of the present invention; and

FIG. 6 is a timeline for a 360° rotation of the cam drive showing radii variations in respective cams during a complete duty cycle of the cam drive.

### DESCRIPTION OF THE PREFERRED EMBODIMENTS

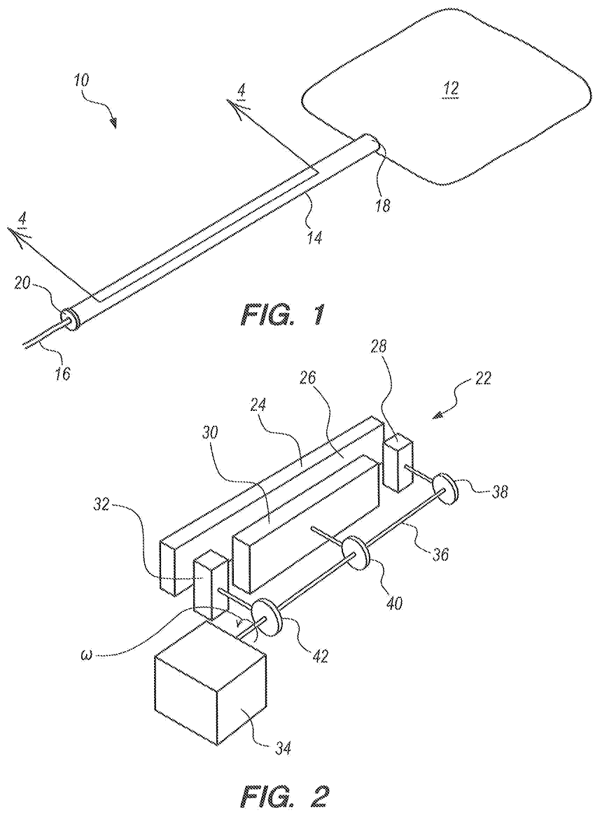

Referring initially to FIG. 1, a disposable component for use in infusing a fluid medicament into a patient (not shown) is shown and is generally designated 10. As shown, the component 10 includes, in combination, a reservoir 12 for holding the fluid medicament, an elastomeric tube 14 and a cannula 16. An upstream end 18 of the elastomeric tube 14 is connected in fluid communication with the reservoir 12. And, a downstream end 20 of the elastomeric tube 14 is connected in fluid communication with the cannula 16.

FIG. 2 shows a mechanical manipulator which is generally designated 22. As shown, the manipulator 22 includes a platen 24 that extends along one side of a receiver channel 26. Aligned along the opposite side of the receiver channel 26 are an upstream pincher 28, a piston 30 and a downstream pincher 32. Note: as used for this disclosure, the terms upstream and downstream indicate the direction in which a fluid medicament flows from the reservoir 12 toward the cannula 16.

Still referring to FIG. 2, it will be seen that the manipulator 22 also includes a torque motor 34 for driving a camshaft 36 in rotation with an angular velocity ω. Shown mounted on the camshaft 36 for rotation therewith are an upstream cam 38, a piston cam 40 and a downstream cam 42. Specifically, in response to a rotation of the camshaft 36 by the torque motor 34, the upstream cam 38 actuates the upstream pincher 28, the piston cam 40 actuates the piston 30 and the downstream cam 42 actuates the downstream pincher 32. As intended for the present invention, a control panel (not shown) can be operated by a user to control w for the purpose of following either a prescribed regimen or a situationally required operation of the manipulator 22, as needed.

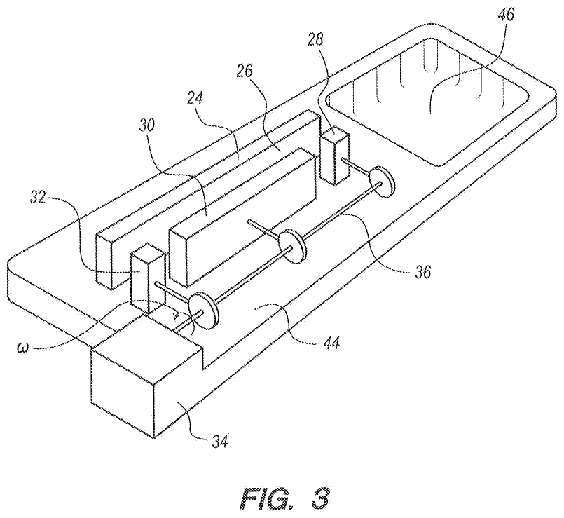

FIG. 3 shows the manipulator 22 mounted on a chassis 44 which is formed with a recess 46. As intended for the present invention, the disposable component 10 is engaged with the manipulator 22 on the chassis 44 for operational purposes. Specifically, with this engagement, the reservoir 12 is positioned in the recess 46 and the elastomeric tube 14 is positioned in the receiver channel 26 against the platen 24. Also, across the receiver channel 26 from the platen 24, the elastomeric tube 14 will be positioned against the upstream pincher 28, the piston 30 and the downstream pincher 32. Further, the cannula 16 will extend from the receiver channel 26 so it can be inserted into the patient for infusion of fluid medicament from the reservoir 12, and into the patient. A cover (not shown) for the chassis 44 can be provided to operationally protect the manipulator 22 and hold the component 10 on the chassis 44 during use.

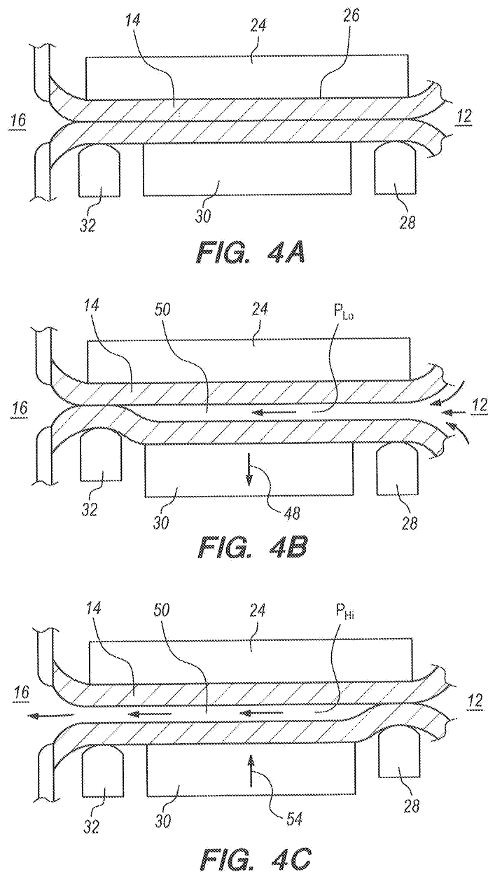

For an operation of the present invention, the cooperation of structure between the elastomeric tube 14 and the manipulator 22 will be best appreciated with reference to FIGS. 4A-C. In detail, FIGS. 4A-C show a sequence of interactive configurations that occur between the elastomeric tube 14 and the manipulator 22 during a duty cycle of the present invention. In overview, FIG. 4A shows a configuration wherein a section of the elastomeric tube 14 is being simultaneously occluded against the platen 24 by actions of the upstream pincher 28, the piston 30 and the downstream pincher 32. The configuration shown in FIG. 4A is a default configuration and will be cyclically repeated at the beginning/end of each duty cycle.

FIG. 4B shows a configuration for the manipulator 22 when the upstream pincher 28 has been opened, and the downstream pincher 32 remains closed. The piston 30 is then withdrawn from the platen 24 in the direction of arrow 48 during a time interval t₁-t₂. The result here is that the elastomeric tube 14 is allowed to expand back to its unstressed condition with an open lumen 50. The consequence of this expansion is that a relatively lower pressure p_{Lo} is created in the elastomeric tube 14, which draws fluid medicament from the reservoir 12 into the lumen 50 of the elastomeric tube 14. This action is hereinafter referred to generally as a filling operation 52. At the time t₂ when the filling operation 52 is ended, the upstream pincher 28 and the downstream pincher 32 execute a changeover operation from an open/close relationship to a close/open relationship.

Following the filling operation 52, and after the changeover operation for the pinchers 28 and 32, FIG. 4C shows a configuration for the manipulator 22 wherein the upstream pincher 28 is closed and the downstream pincher 32 has opened. The piston 30 is then incrementally advanced in the direction of arrow 54 toward the platen 24 to create a relatively high pressure p_{Hi} in the lumen 50 during a time interval t₂-t₃. Thus, liquid medicament is pumped from the lumen 50 and through the cannula 16 for infusion into the patient during what is hereinafter referred to as a pumping operation 56. Together, the filling operation 52 and the pumping operation 56 occur on a timeline t₁₋₄, wherein:
t₀-t₁ is a time interval when the upstream pincher 28, piston 30 and downstream pincher 32 are all closed to begin a duty cycle;
t₁-t₂ is a time interval when the downstream pincher 32 remains closed while the upstream pincher 28 is opened and the piston 30 is withdrawn to create p_{Lo} in the lumen 50 to draw fluid medicament into the tube 14 through the upstream end 18 of the tube 14;
t₂ is a time for the pincher 28/32 changeover operation;
t₂-t₃ is a time interval when the upstream pincher 28 is closed and the downstream pincher 32 is open as the piston 30 is advanced to create p_{Hi} in the lumen 50 to expel fluid medicament from the tube 14 through the downstream end 20 of the tube 14;
t₃-t₄ is a time interval when both the upstream and the downstream pinchers 28/32 are closed to end the duty cycle; and
t₄=₀ to begin a new duty cycle.

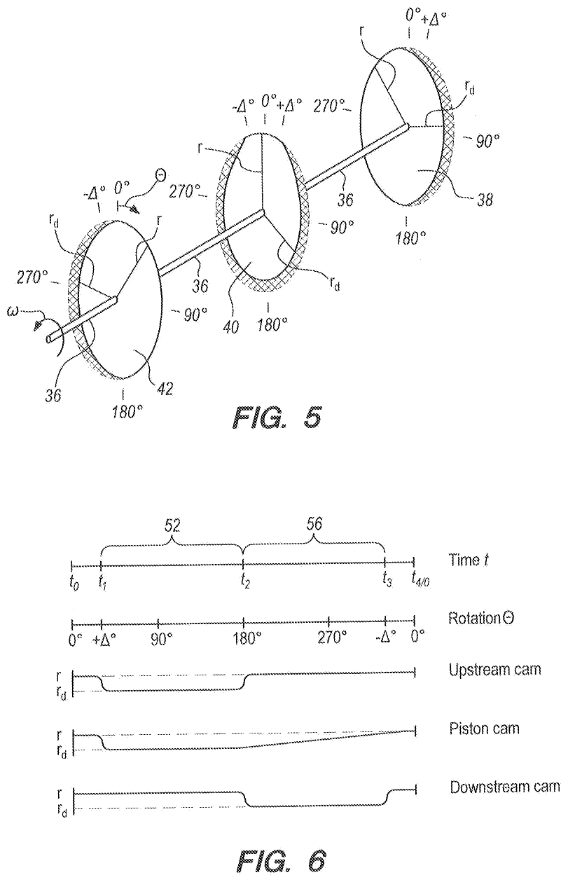

FIG. 5 and FIG. 6 are cross referenced for the disclosure of an operation of the present invention during a duty cycle. As shown in FIG. 5 the camshaft 36 is being rotated counterclockwise at the velocity ω, which can be varied or held constant, as desired. As also shown in FIG. 5, the material that has been removed at the periphery of the respective cams 38, 40 and 42 to reduce a common radius r to a respectively diminished radii r_{d} have been crosshatched to indicate the removal of material that is needed to create the cam. In FIG. 6, these variations in radii are shown in their angular relationship θ with each other over a duty cycle timeline, t₁-t₃, including an occlusion interval t_{4/0}.

Additional aspects of the present invention that are noteworthy include the fact that the present invention is envisioned as an insulin pump for the treatment of diabetes. Accordingly, the torque motor 34 can be manually controlled, or preprogrammed, according to the needs of the patient/user. Further, the component 10 is envisioned as being disposable. Moreover, the entire combination of components and mechanisms that constitute the present invention are envisioned as being transportable with the patient/user for an operation of the present invention for constant use by the patient/user.

While the particular Portable Infusion Pump with Pinch/Squeeze Pumping Action as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for infusing a fluid medicament into a patient which comprises:
a collapsible reservoir for holding the fluid medicament;
a cannula;
a hollow, elastomeric tube formed with a lumen and having an upstream end connected in fluid communication with the reservoir of fluid medicament and a downstream end connected in fluid communication with the cannula; and
a manipulator adapted for engagement with a tube section of the elastomeric tube between the upstream end and the downstream end of the elastomeric tube, wherein the tube section is manipulated to create a low fluid pressure, p_{Lo}, at the upstream end of the elastomeric tube to draw fluid medicament into the tube section from the reservoir and, alternately, to create a high fluid pressure, p_{Hi}, at the downstream end of the elastomeric tube to push fluid medicament through the cannula from the tube section to infuse fluid medicament into the patient.

2. The system of claim 1 further comprising a chassis adapted to receive and hold the elastomeric tube, and wherein the manipulator is mounted on the chassis for engagement with the tube section of the elastomeric tube.

3. The system of claim 2 wherein the manipulator creates a reciprocating constrict/dilate action along the tube section, and comprises:
a platen mounted on the chassis;
an upstream pincher mounted on the chassis and located adjacent the reservoir;
a downstream pincher mounted on the chassis and located adjacent the cannula, wherein the upstream and downstream pinchers act in concert with each other in a close/open and open/close changeover operation to individually and separately occlude the elastomeric tube against the platen; and
a piston mounted on the chassis to provide a reciprocating motion against a portion of the tube section located between the upstream and downstream pinchers, wherein the piston is advanced toward the platen when the downstream pincher is open, to constrict the lumen of the tube section therebetween and create a high pressure pH, to push fluid medicament from the tube section and through the cannula, and wherein the piston is alternately withdrawn from the platen when the upstream pincher is open, to dilate the lumen of the tube section and create a low pressure p_{Lo} to draw fluid medicament from the reservoir and into the tube section.

4. The system of claim 3 wherein the tube section of the elastomeric tube is memory biased to return to a configuration with a dilated lumen, and wherein the lumen of the tube section is increasingly dilated as the piston is being withdrawn.

5. The system of claim 4 wherein each close/open and open/close changeover operation is accomplished after a predetermined time interval when the upstream pincher and the downstream pincher are both closed.

6. The system of claim 5 wherein p_{Hi} is greater than p_{Lo} (p_{Hi}>p_{Lo}) and the difference between p_{Hi} and p_{Lo} is less than 5 psi (p_{Hi}-p_{Lo}<5 psi).

7. The system of claim 3 wherein during a duty cycle, the piston withdraws when the upstream pincher and the downstream pincher are respectively open/close, and the piston advances when the upstream pincher and the downstream pincher are respectively close/open in accordance with a timeline t₁₋₄, wherein:
t₀-t₁ is a time interval when both the upstream and the downstream pinchers are closed to begin the duty cycle;
t₁-t₂ is a time interval when the downstream pincher remains closed while the upstream pincher is opened and the piston is withdrawn to create p_{Lo} in the lumen to draw fluid medicament into the tube through the upstream end of the tube;
t₂ is a time for the pincher changeover operation;
t₂-t₃ is a time interval when the upstream pincher is now closed and the downstream pincher is open as the piston is advanced to create p_{Hi} in the lumen to expel fluid medicament from the tube through the downstream end of the tube;
t₃-t₄ is a time interval when both the upstream and the downstream pinchers are closed to end the duty cycle; and
t₄=t₀ to begin a new duty cycle.

8. The system of claim 7 further comprising:
a camshaft mounted on the chassis for rotation around an axis;
a piston cam affixed to the camshaft and positioned on the chassis for cooperation with the piston during the duty cycle to withdraw the piston from the tube during the time interval t₁-t₂ and to advance the piston toward the tube during the time interval t₂-t₃;
an upstream cam affixed to the camshaft and positioned on the chassis for cooperation with the upstream pincher to open the upstream pincher during the time interval t₁-t₂;
a downstream cam affixed to the camshaft and positioned on the chassis for cooperation with the downstream pincher to open the downstream pincher during the time interval t₂-t₃; and
a torque motor mounted on the chassis and engaged with the camshaft for simultaneously rotating the piston cam, the upstream cam and the downstream cam during the duty cycle.

9. The system of claim 7 wherein each duty cycle has a time duration in the range of 1-5 minutes and the fluid medicament is insulin.

10. A system for infusing a fluid medicament into a patient during a duty cycle which comprises:
a chassis;
a platen mounted on the chassis;
an upstream pincher mounted on the chassis and located adjacent a reservoir;
a downstream pincher mounted on the chassis and located adjacent a cannula, wherein the upstream and downstream pinchers interact with each other in a close/open and open/close changeover operation to individually occlude an elastomeric tube against the platen; and
a piston mounted on the chassis to provide a reciprocating motion against a portion of a tube section located between the upstream and downstream pinchers, wherein the piston is advanced toward the platen when the downstream pincher is open, to constrict the lumen of the tube section therebetween and create a high pressure p_{Hi} to push fluid medicament from the tube section and through the cannula, and wherein the piston is alternately withdrawn from the platen when the upstream pincher is open, to dilate the lumen of the tube section and create a low pressure p_{Lo} to draw fluid medicament from the reservoir and into the tube section.

11. The system of claim 10 wherein during the duty cycle, the piston withdraws when the upstream pincher and the downstream pincher are respectively open/close, and the piston advances when the upstream pincher and the downstream pincher are respectively close/open.

12. The system of claim 11 wherein the duty cycle is completed within a time duration t₁₋₄, wherein:
t₀-t₁ is a time interval when both the upstream and the downstream pinchers are closed to begin the duty cycle;
t₁-t₂ is a time interval when the downstream pincher remains closed while the upstream pincher is opened and the piston is withdrawn to create p_{Lo} in the lumen to draw fluid medicament into the tube through the upstream end of the tube;
t₂ is a time for the pincher changeover operation;
t₂-t ₃ is a time interval when the upstream pincher is now closed and the downstream pincher is open as the piston is advanced to create p_{Hi} in the lumen to expel fluid medicament from the tube through the downstream end of the tube;
t₃-t₄ is a time interval when both the upstream and the downstream pinchers are closed to end the duty cycle; and
t₄=t₀ to begin a new duty cycle.

13. The system of claim 12 further comprising:
a camshaft mounted on the chassis for rotation around an axis;
a piston cam affixed to the camshaft and positioned on the chassis for cooperation with the piston during the duty cycle to allow the withdrawal of the piston from the tube during the time interval t₁-t₂ and to advance the piston toward the tube during the time interval t₂-t₃;
an upstream cam affixed to the camshaft and positioned on the chassis for cooperation with the upstream pincher to open the upstream pincher during the time interval t₁-t₂;
a downstream cam affixed to the camshaft and positioned on the chassis for cooperation with the downstream pincher to open the downstream pincher during the time interval t₂-t₃; and
a torque motor mounted on the chassis and engaged with the camshaft for simultaneously rotating the piston cam, the upstream cam and the downstream cam during a duty cycle.

14. The system of claim 10 wherein p_{Hi} is greater than p_{Lo} (p_{Hi}>p_{Lo}) and the difference between p_{Hi} and P_{Lo} is less than 5 psi (P_{Hi}-P_{Lo}<5 psi).

15. The system of claim 10 wherein each close/open and open/close changeover operation is accomplished after a predetermined time interval when the upstream pincher and the downstream pincher are both closed.

16. The system of claim 10 wherein each duty cycle has a time duration in the range of 1-5 minutes and the fluid medicament is insulin.

17. A method for operating a device to infuse a fluid medicament into a patient which comprises the steps of:
providing a disposable component comprising a collapsible reservoir for holding the fluid medicament, a cannula, and an elastomeric tube formed with a lumen and having an upstream end connected in fluid communication with the reservoir of fluid medicament and a downstream end connected in fluid communication with the cannula;
engaging a section of the tube with a manipulator between the upstream end and the downstream end of the elastomeric tube; and
operating the manipulator in accordance with a predetermined protocol to create a low fluid pressure, p_{Lo}, at the upstream end of the elastomeric tube to draw fluid medicament into the tube section from the reservoir, and to create a high fluid pressure, p_{Hi}, at the downstream end of the elastomeric tube to push fluid medicament through the cannula from the tube section to infuse fluid medicament into the patient.

18. The method of claim 17 wherein the predetermined protocol establishes a plurality of sequential duty cycle operations wherein each duty cycle has a timeline t₁₋₄, for fluid pressure variations in the tube section, wherein:
t₀-t₁ is a time interval when both an upstream pincher and a downstream pincher are closed to begin a duty cycle;
t₁-t₂ is a time interval when the downstream pincher remains closed while the upstream pincher is opened and a piston is withdrawn to create p_{Lo} in the lumen to draw fluid medicament into the tube through the upstream end of the tube;
t₂ is a time for a pincher changeover operation;
t₂-t₃ is a time interval when the upstream pincher is now closed and the downstream pincher is open as the piston is advanced to create p_{Hi} in the lumen to expel fluid medicament from the tube through the downstream end of the tube;
t₃-t₄ is a time interval when both the upstream and the downstream pinchers are closed to end the duty cycle; and
t₄=t₀ to begin a new duty cycle.

19. The method of claim 18 wherein the time interval t₁-t₂ is equal to the time interval t₂-t₃.

20. The method of claim 18 wherein p_{Lo} and p_{Hi} are variable within their respective time intervals.

21. A method for operationally assembling an intravenous infusion pump to infuse a fluid medicament into a user which comprises the steps of:
acquiring a portable infusion pump, wherein the infusion pump includes a chassis formed with a recess and has a manipulator mounted on the chassis, wherein the manipulator is formed with a receiver channel;
obtaining a disposable unit containing the fluid medicament to be infused, wherein the disposable unit includes a hollow, elastomeric tube having an upstream end connected in fluid communication with a collapsible reservoir holding the fluid medicament, and a downstream end connected in fluid communication with a cannula;
engaging a section of the elastomeric tube of the disposable unit between the upstream end and the downstream end of the elastomeric tube with the receiver channel of the manipulator, for manipulation of the tube section by the manipulator to create a low fluid pressure, p_{Lo}, at the upstream end of the elastomeric tube to draw fluid medicament into the tube section from the reservoir and, alternately, to create a high fluid pressure, p_{Hi}, at the downstream end of the elastomeric tube; and
inserting the cannula into body tissue of a user.

22. The method of claim 21 wherein the manipulator creates a reciprocating constrict/dilate action along the tube section, and comprises:
a platen mounted on the chassis;
an upstream pincher mounted on the chassis and located adjacent the reservoir;
a downstream pincher mounted on the chassis and located adjacent the cannula, wherein the upstream and downstream pinchers act in concert with each other in a close/open and open/close changeover operation to individually and separately occlude the elastomeric tube against the platen; and
a piston mounted on the chassis to provide a reciprocating motion against a portion of the tube section located between the upstream and downstream pinchers, wherein the piston is advanced toward the platen when the downstream pincher is open, to constrict the lumen of the tube section therebetween and create a high pressure p_{Hi} to push fluid medicament from the tube section and through the cannula, and wherein the piston is alternately withdrawn from the platen when the upstream pincher is open, to dilate the lumen of the tube section and create a low pressure p_{Lo} to draw fluid medicament from the reservoir and into the tube section.

23. The method of claim 22 wherein during a duty cycle, the piston withdraws when the upstream pincher and the downstream pincher are respectively open/close, and the piston advances when the upstream pincher and the downstream pincher are respectively close/open in accordance with a timeline t₁₋₄, wherein:
t₀-t₁ is a time interval when both the upstream and the downstream pinchers are closed to begin the duty cycle;
t₁-t₂ is a time interval when the downstream pincher remains closed while the upstream pincher is opened and the piston is withdrawn to create p_{Lo} in the lumen to draw fluid medicament into the tube through the upstream end of the tube;
t₂ is a time for the pincher changeover operation;
t₂-t₃ is a time interval when the upstream pincher is now closed and the downstream pincher is open as the piston is advanced to create p_{Hi} in the lumen to expel fluid medicament from the tube through the downstream end of the tube;
t₃-t₄ is a time interval when both the upstream and the downstream pinchers are closed to end the duty cycle; and
t₄=t₀ to begin a new duty cycle.

24. The method of claim 23 wherein the manipulator further comprises:
a camshaft mounted on the chassis for rotation around an axis;
a piston cam affixed to the camshaft and positioned on the chassis for cooperation with the piston during the duty cycle to withdraw the piston from the tube during the time interval t₁-t₂ and to advance the piston toward the tube during the time interval t₂-t₃;
an upstream cam affixed to the camshaft and positioned on the chassis for cooperation with the upstream pincher to open the upstream pincher during the time interval t₁-t₂;
a downstream cam affixed to the camshaft and positioned on the chassis for cooperation with the downstream pincher to open the downstream pincher during the time interval t₂-t₃; and
a torque motor mounted on the chassis and engaged with the camshaft for simultaneously rotating the piston cam, the upstream cam and the downstream cam during the duty cycle.

25. The method of claim 24 wherein each duty cycle has a time duration in the range of 1-5 minutes and the fluid medicament is insulin, and wherein p_{Hi} is greater than p_{Lo} (p_{Hi}>p_{Lo}) and the difference between p_{Hi} and p_{Lo} is less than 5 psi (p_{Hi}-p_{Lo}<5 psi).
